(19) United States
(12) Patent Application Publication
(10) Pub. No.: US 2026/0221262 A1
(43) Pub. Date: Jul. 30, 2026

(54) ARTIFICIAL INTELLIGENCE TECHNOLOGIES FOR DETERMINING PATIENT-SPECIFIC THERAPEUTIC ACTION ITEMS

(71) Applicant: HELLOJOY, INC., Chicago, IL (US)

(72) Inventors: Daniel Freed, Chicago, IL (US); Roger Deetz, Indianapolis, IN (US); Chris Cutter, Berkely, CA (US); Alex Bartling, Toronto (CA); John Romas, Bloomfield Hills, MI (US); Vivian Chung Easton, Los Angeles, CA (US); Eric Troyer, Indianapolis, IN (US)

(21) Appl. No.: 19/038,978

(22) Filed: Jan. 28, 2025

(51) Int. Cl. G16H 20/70; G10L 17/02; G10L 21/028; G10L 25/66; G16H 10/60; G16H 50/30

(52) U.S. Cl. CPC G16H 20/70; G10L 17/02; G10L 21/028; G10L 25/66; G16H 10/60; G16H 50/30

(57) ABSTRACT

Technologies for determining patient-specific therapeutic action items include a computing system configured to obtain audio data indicative of recorded audio associated with a mental health therapy session between a clinician and a patient and transcribe the obtained audio data to produce a diarized transcript indicative of words spoken during the therapy session between the clinician and the patient. The computing system is also configured to generate, with an artificial intelligence model, treatment data including suggestion data, indicative of one or more suggestions for corresponding action items to advance mental health treatment for the patient based on the diarized transcript, analyze the treatment data to select one or more of the suggestions to present to the clinician to advance the mental health treatment for the patient, and present the selected one or more suggestions to the clinician to advance the mental health treatment for the patient.

### BACKGROUND

In the United States and other countries, mental health disorders have gained increased attention over time, with the demand for clinicians who are qualified to provide therapy for mental health patients increasing dramatically as a result. With the increased demand and influx of mental health patients, a typical clinician may be overwhelmed by the amount of work that must be performed before and after a therapy session with each mental health patient while also making themselves available for interactions with other patients. Accordingly, clinicians may face the prospect of decreasing the quality of mental health care provided to each patient in order to accommodate a higher number of patients or, conversely, reducing their availability, through a reduced number of appointments, to provide mental health care while maintaining a higher degree of care for each individual patient that is able to schedule an appointment with the clinician. As will be appreciated, neither scenario provides an efficient solution to the growing demand for mental health assistance.

### SUMMARY

One embodiment is directed to a unique system, components, and methods for determining, with artificial intelligence, action items to advance the mental health treatment for patients. Other embodiments are directed to apparatuses, systems, devices, hardware, methods, and combinations thereof for determining patient-specific therapeutic action items.

According to an embodiment, a method for determining patient-specific therapeutic action items may include obtaining, by a computing system, audio data indicative of recorded audio associated with a mental health therapy session between a clinician and a patient. The method may also include transcribing, by the computing system, the obtained audio data to produce a diarized transcript indicative of words spoken during the therapy session between the clinician and the patient. Additionally, the method may include generating, by the computing system and with an artificial intelligence model, treatment data including suggestion data. The suggestion data may be indicative of one or more suggestions for corresponding action items to advance mental health treatment for the patient based on the diarized transcript. The method may also include analyzing, by the computing system, the treatment data to select one or more of the suggestions to present to the clinician to advance the mental health treatment for the patient. Further, the method may include presenting, by the computing system, the selected one or more suggestions to the clinician to advance the mental health treatment for the patient.

In some embodiments, obtaining audio data comprises obtaining audio data from a user device of the clinician.

In some embodiments, transcribing the obtained audio data comprises producing the diarized transcript that partitions the words spoken during the therapy session based on a speaker identity.

In some embodiments, generating the treatment data comprises generating, with a large language model, a clinical analysis.

In some embodiments, generating the treatment data comprises generating, with a large language model, a treatment plan.

In some embodiments, generating the treatment data comprises generating, with a large language model, one or more assessment suggestions, wherein each assessment suggestion is indicative of an action item relating to an assessment of the patient.

In some embodiments, generating the treatment data comprises generating, with a large language model, one or more worksheet suggestions, wherein each worksheet suggestion is indicative of an action item relating to a worksheet to be completed by the patient.

In some embodiments, generating the treatment data comprises generating, with a large language model, one or more intervention suggestions, wherein each intervention suggestion is indicative of an intervention to be performed relative to the patient.

In some embodiments, generating the treatment data comprises generating a progress note indicative of a progress relative to the mental health of the patient.

In some embodiments, generating the treatment data comprises generating preparation materials for a subsequent therapy session between the clinician and the patient.

In some embodiments, analyzing the treatment data comprises comparing the one or more suggestions to exclusion criteria to filter out at least one of the one or more suggestions based on factors specific to the patient.

In some embodiments, analyzing the treatment data comprises determining a confidence score associated with each suggestion and selecting at least one of the suggestions associated with a confidence score that satisfies a target confidence score for presentation to the clinician.

In some embodiments, generating treatment data comprises generating preparation materials for a subsequent therapy session between the clinician and the patient, and the method further includes presenting, by the computing system and to the clinician, the preparation materials for the subsequent therapy session.

In some embodiments, the method may further include utilizing a message queue service to queue a serverless function to generate a clinical analysis from the diarized transcript.

In some embodiments, the method may further include utilizing a message queue service to queue a serverless function to generate a treatment plan from the diarized transcript.

In some embodiments, the method may further include utilizing a message queue service to queue a serverless function to generate a treatment plan from the diarized transcript and demographic information associated with the patient.

In some embodiments, the method may additionally comprise utilizing a message queue service to queue a serverless function to generate the one or more suggestions from the diarized transcript.

In some embodiments, the method may additionally comprise utilizing patient context information indicative of one or more of an accepted treatment plan, a diagnosis and focus of treatment, a transcript prior to the diarized transcript, or a clinical assessment of the patient to generate the one or more suggestions with the serverless function.

In some embodiments, the method may additionally comprise utilizing a library of worksheets and patient context information indicative of an accepted treatment plan associated with the patient to generate the one or more suggestions with the serverless function, wherein the one or more suggestions are indicative of one or more worksheets to be completed by the patient.

In some embodiments, the method may additionally comprise utilizing patient context information indicative of one or more of an accepted treatment plan associated with the patient, a diagnosis and focus of treatment, a transcript prior to the diarized transcript, or an intervention that was previously performed relative to the patient, to generate the one or more suggestions with the serverless function, wherein the one or more suggestions are indicative of one or more interventions to be performed with the patient.

In some embodiments, the method may further include utilizing a message queue service to queue a serverless function to generate a progress note that is based on the diarized transcript and patient context information indicative of one or more of a treatment plan associated with the patient, demographic information associated with the patient, or a clinician preference in a multiple step workflow that utilizes a large language model.

According to another embodiment, a computing system for determining patient-specific therapeutic action items may include at least one processor and at least one memory comprising a plurality of instructions stored thereon that, in response to execution by the at least one processor, causes the computing system to obtain audio data indicative of recorded audio associated with a mental health therapy session between a clinician and a patient. The instructions may also cause the computing system to transcribe the obtained audio data to produce a diarized transcript indicative of words spoken during the therapy session between the clinician and the patient. Further, the instructions may cause the computing system to generate, with an artificial intelligence model, treatment data including suggestion data, indicative of one or more suggestions for corresponding action items to advance mental health treatment for the patient based on the diarized transcript. Additionally, the instructions may cause the computing system to analyze the treatment data to select one or more of the suggestions to present to the clinician to advance the mental health treatment for the patient. Additionally, the instructions may cause the computing system to present the selected one or more suggestions to the clinician to advance the mental health treatment for the patient.

In some embodiments, to obtain audio data comprises to obtain audio data from a user device of the clinician.

In some embodiments, to transcribe the obtained audio data comprises to produce the diarized transcript that partitions the words spoken during the therapy session based on a speaker identity.

In some embodiments, to generate the treatment data comprises to generate, with a large language model, a clinical analysis.

In some embodiments, to generate the treatment data comprises to generate, with a large language model, a treatment plan.

In some embodiments, to generate the treatment data comprises to generate, with a large language model, one or more assessment suggestions, wherein each assessment suggestion is indicative of an action item relating to an assessment of the patient.

In some embodiments, to generate the treatment data comprises to generate, with a large language model, one or more worksheet suggestions, wherein each worksheet suggestion is indicative of an action item relating to a worksheet to be completed by the patient.

In some embodiments, to generate the treatment data comprises to generate, with a large language model, one or more intervention suggestions, wherein each intervention suggestion is indicative of an intervention to be performed relative to the patient.

In some embodiments, to generate the treatment data comprises to generate a progress note indicative of a progress relative to the mental health of the patient.

In some embodiments, to generate the treatment data comprises to generate preparation materials for a subsequent therapy session between the clinician and the patient.

In some embodiments, to analyze the treatment data comprises to compare the one or more suggestions to exclusion criteria to filter out at least one of the one or more suggestions based on factors specific to the patient.

In some embodiments, to analyze the treatment data comprises to determine a confidence score associated with each suggestion and select at least one of the suggestions associated with a confidence score that satisfies a target confidence score for presentation to the clinician.

In some embodiments, to generate treatment data comprises to generate preparation materials for a subsequent therapy session between the clinician and the patient, and the instructions may additionally cause the computing system to present, to the clinician, the preparation materials for the subsequent therapy session.

In some embodiments, the instructions additionally cause the computing system to utilize a message queue service to queue a serverless function to generate a clinical analysis from the diarized transcript.

In some embodiments, the instructions additionally cause the computing system to utilize a message queue service to queue a serverless function to generate a treatment plan from the diarized transcript.

In some embodiments, the instructions additionally cause the computing system to utilize a message queue service to queue a serverless function to generate a treatment plan from the diarized transcript and demographic information associated with the patient.

In some embodiments, the instructions additionally cause the computing system to utilize a message queue service to queue a serverless function to generate the one or more suggestions from the diarized transcript.

In some embodiments, the instructions additionally cause the computing system to utilize patient context information indicative of one or more of an accepted treatment plan, a diagnosis and focus of treatment, a transcript prior to the diarized transcript, or a clinical assessment of the patient to generate the one or more suggestions with the serverless function.

In some embodiments, the instructions additionally cause the computing system to utilize a library of worksheets and patient context information indicative of an accepted treatment plan associated with the patient to generate the one or more suggestions with the serverless function, wherein the one or more suggestions are indicative of one or more worksheets to be completed by the patient.

In some embodiments, the instructions additionally cause the computing system to utilize patient context information indicative of one or more of an accepted treatment plan associated with the patient, a diagnosis and focus of treatment, a transcript prior to the diarized transcript, or an intervention that was previously performed relative to the patient, to generate the one or more suggestions with the serverless function, wherein the one or more suggestions are indicative of one or more interventions to be performed with the patient.

In some embodiments, the instructions additionally cause the computing system to utilize a message queue service to queue a serverless function to generate a progress note that is based on the diarized transcript and patient context information indicative of one or more of a treatment plan associated with the patient, demographic information associated with the patient, or a clinician preference in a multiple step workflow that utilizes a large language model.

This summary is not intended to identify key or essential features of the claimed subject matter, nor is it intended to be used as an aid in limiting the scope of the claimed subject matter. Further embodiments, forms, features, and aspects of the present application shall become apparent from the description and figures provided herewith.

### BRIEF DESCRIPTION OF THE DRAWINGS

The concepts described herein are illustrative by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements.

FIG. 1 depicts a simplified block diagram of at least one embodiment of a system for determining patient-specific therapeutic action items with artificial intelligence;

FIG. 2 is a simplified block diagram of at least one embodiment of a computing device;

FIGS. 3-4 are a simplified flow diagram of at least one embodiment of a method for determining patient-specific therapeutic action items;

FIG. 5 is a diagram of at least one embodiment of a pipeline that may be utilized by the system of FIG. 1 for audio capture;

FIG. 6 is a diagram of at least one embodiment of a pipeline that may be utilized by the system of FIG. 1 for transcription;

FIG. 7 is a diagram of at least one embodiment of a pipeline that may be utilized by the system of FIG. 1 for progress note and treatment plan generation;

FIG. 8 is a diagram of at least one embodiment of a pipeline that may be utilized by the system of FIG. 1 for identifying suggestions to present to a clinician; and

FIGS. 9-13 are diagrams of embodiments of user interfaces that may be presented by the system of FIG. 1.

### DETAILED DESCRIPTION

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described.

Further, particular features, structures, or characteristics may be combined in any suitable combinations and/or sub-combinations in various embodiments.

Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

The disclosed embodiments may, in some cases, be implemented in hardware, firmware, software, or a combination thereof. The disclosed embodiments may also be implemented as instructions carried by or stored on one or more transitory or non-transitory machine-readable (e.g., computer-readable) storage media, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may not be included or may be combined with other features.

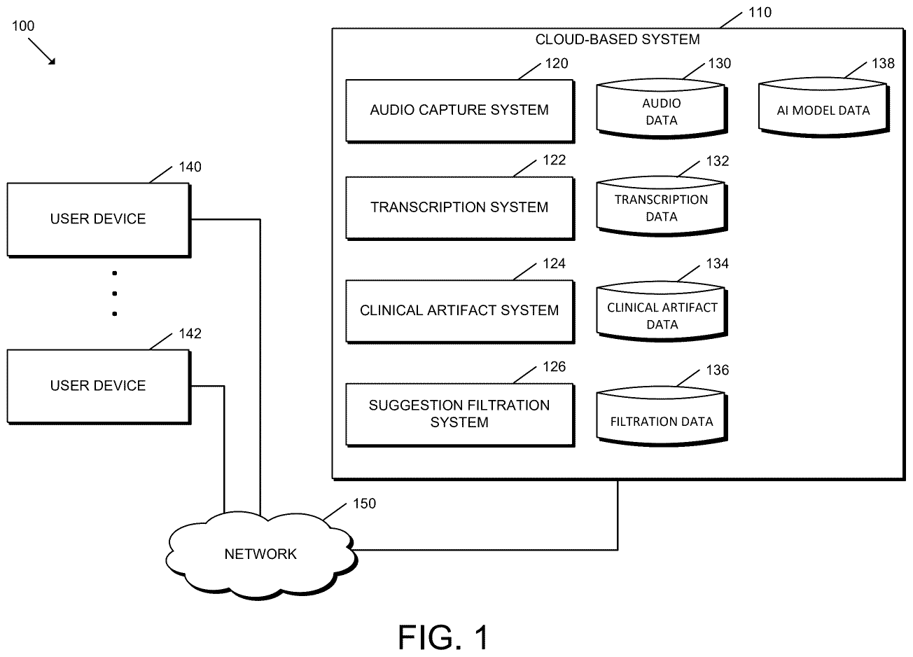

Referring now to FIG. 1, a system 100 (a computing system) for determining patient-specific therapeutic action items to advance mental health treatment for patients includes a cloud-based system 110, a set of user devices 140, 142, and a network 150. The illustrative cloud-based system 110 includes an audio capture system 120, a transcription system 122, a clinical artifact system 124, a suggestion filtration system 126, audio data 130, transcription data 132, clinical artifact data 134, filtration data 136, and artificial intelligence model data 138. Although only one audio capture system 120, one transcription system 122, one clinical artifact system 124, and one suggestion filtration system 126, two user devices 140, 142, and one network 150 are shown in the illustrative embodiment of FIG. 1, the system 100 may include any number of audio capture systems 120, transcription systems 122, clinical artifact systems 124, suggestion filtration systems 126, user devices 140, 142, and networks 150. For example, in some embodiments, multiple cloud-based systems 110 (e.g., related or unrelated systems) may be used to perform the various functions described herein. Further, in some embodiments, one or more of the systems described herein may be excluded from the system 100, one or more of the systems described as being independent may form a portion of another system, and/or one or more of the systems described as forming a portion of another system may be independent.

The cloud-based system 110 may be embodied as any one or more types of devices/systems capable of performing the functions described herein. For example, as described herein, the audio capture system 120 is configured to obtain a recording of a therapy session between a clinician associated with a user device 140, 142 and a patient via a corresponding connection through the network 150 and store the recording as the audio data 130, such as in blob storage, for subsequent processing. In doing so, the audio capture system 120 may interact with a client-side application executed by the user device 140, 142, such as a web browser, to serve content, such as instructions executable within the web browser, to access a microphone of the user device 140, 142, record segments of the audio session, and transmit those segments to the audio capture system 120 for storage as the audio data 130. The segments may be embodied as contiguous sets of audio data, having a predefined length, such as five seconds. In at least some embodiments, the instructions served to the user device 140, 142 cause the user device 140, 142 to create a local queue of the audio segments to upload to the audio capture system 120 when possible. The creation and use of a local queue guards against the possibility of losing segments of the audio if a connection between the user device 140, 142 and the audio capture system 120 is intermittent.

The transcription system 122 may combine the audio segments in the audio data 130 that are associated with a given therapy session into a single continuous recording of the therapy session. Further, the transcription system 122, in the illustrative embodiment, generates a transcript of the recording of the therapy session. In doing so, the transcription system 122 may perform speech recognition to identify the words that were spoken during the therapy session. To do so, the transcription system 122, in at least some embodiments, performs feature extraction to identify features or characteristics of sections of the audio that are usable to identify phonemes and words. Those features may include mel-frequency cepstral coefficients (MFCCs). MFCCs approximate the operations of the human auditory system by filtering an audio signal based on a mel scale in which frequencies are perceived logarithmically at relatively high frequencies and linearly at low frequencies. In the mel-frequency cepstrum, a short-term power spectrum of a sound is represented, based on a linear cosine transform of a log power spectrum on the nonlinear mel scale of frequency. The MFCCs may represent coefficients that constitute the MFC. In at least some embodiments, the transcription system 122 may perform a Fourier transform of the audio signal and map powers of the spectrum obtained from the Fourier transform to the mel scale, using overlapping windows, such as triangular or cosine overlapping windows. Further the transcription system 122 may determine logs of the powers at each of the mel frequencies, determine the discrete cosine transform of the set of mel log powers, and define the MFCCs as the amplitudes of the resulting spectrum. Other features that the transcription system 122 may determine include a zero crossing rate and/or a pitch. The zero crossing rate may represent a number of times an audio signal crosses a zero axis and the pitch may represent the fundamental frequency of a voice.

The transcription system 122 may also generate feature vectors based on the features determined from the audio recording. Each feature vector may be embodied as a set of values that represent the features in a numeric form to be utilized in subsequent algorithms designed to operate on numeric values rather than qualitative information. The transcription system 122 may also utilize a decoder which employs one or more acoustic models, a pronunciation dictionary, and one or more language models to determine the words that are spoken in the recorded audio. In doing so, the transcription system 122 may perform natural language processing (NLP), using hidden Markov models (HMM), N-grams, neural networks, and/or a combination thereof. That is, in some embodiments, the transcription system 122 utilizes hidden Markov models in which observations are dependent on a hidden or latent Markov events, such as tags indicative of parts of speech, to determine a probability of a next unit (e.g., a word, syllable, sentence, etc.) in a sequence of recorded audio. In some embodiments, the transcription system 122 utilizes a language model that assigns probabilities to n-grams, which are sequences of a number (e.g., n) of words, based on statistics.

The transcription system 122 may additionally or alternatively utilize a neural network, such as a recurrent neural network (RNN), that operates on a continuous representation or embedding of words as a non-linear combination of weights. A neural network, also referred to herein as an artificial neural network, is a set of connected units or nodes that model the neurons in a brain and that are connected via edges, which model synapses in the brain. Each neuron is configured to receive corresponding signals from connected neurons, then process those signals and produce a resulting signal to other connected neurons. The resulting signal is produced based on an activation function, which is a function that determines an output of a node based on the individual inputs and weights associated with those inputs. The activation function may be, for example, a rectified linear unit activation function, a gaussian error linear unit activation function, or a logistic sigmoid function. An RNN is a specialized type of artificial neural network designed for sequential data processing and that utilizes a recurrent unit that maintains a hidden state that is updated for each of multiple time steps based on a present input and a previous hidden state. A feedback loop may enable the RNN to learn from previous inputs and incorporate that information into the current processing.

Further, the transcription system 122, in the illustrative embodiment, performs speaker diarization. In performing speaker diarization, the transcription system 122 identifies speakers and segments the speech represented in the audio recording by speaker identity, to assist in distinguishing the clinician from the patient. The transcription system 122, in the illustrative embodiment, produces a transcript from the audio recording that indicates the speaker associated with each set of words spoken during the corresponding therapy session and stores the transcript in the transcription data 132.

Based at least in part on the transcription data 132, and in particular the diarized transcript produced by the transcription system 122, the clinical artifact system 124 may generate a clinical analysis using an artificial intelligence model, such as a large language model (LLM). A large language model is a machine learning model designed for natural language processing operations and is trained using self-supervised learning on a relatively large amount of text. In at least some embodiments, a large language model that may be utilized by the clinical artifact system 124 is a generative pretrained transformer that may be fine-tuned through prompt engineering. A generative pre-trained transformer (GPT) is a type of generative artificial intelligence framework based on a transformer deep learning architecture that is pre-trained on a relatively large data set of unlabeled text to produce human-like outputs. In a transformer architecture, text is converted into a vector structure through a word embedding table, and in each of multiple layers of the architecture, the transformer contextualizes the token within the scope of a context window with other tokens through a parallel multi-head attention mechanism. Through the architecture, a signal for a key (e.g., significant) token may be amplified and the signal for less significant token may be de-emphasized. Among other benefits, the transformer architecture enables shorter training times compared to the training times of RNN's for similar tasks.

The clinical artifact system 124 may also generate a treatment plan for the patient based on the transcript, any other available transcripts associated with the patient, and potentially other information regarding the patient that the clinical artifact system 124 may request from the clinician (e.g., via the user device 140, 142). Further, the clinical artifact system 124 may generate suggestions indicative of action items that will enable the patient to progress through the treatment (e.g., the treatment plan). Those suggestions may include assessment suggestions, worksheet suggestions, and, in some embodiments, intervention suggestions. Further, the clinical artifact system 124 may produce one or more progress notes, each of which is indicative of the present progress of the patient through the treatment plan. The clinical artifact system 124 may also produce preparation materials to prepare the clinician for a subsequent therapy session with the patient. The above operations, which, in the illustrative embodiments, utilize an artificial intelligence model, such as a large language model, are described in more detail herein. The clinical artifact system 124, in the illustrative embodiment, stores the clinical analysis, treatment plan, suggestions, progress notes, and preparation materials as the clinical artifact data 134.

The suggestion filtration system 126 may perform filtering operations on the suggestions from the clinical artifact data 134 to select suggestions that satisfy particular criteria. For example, the suggestion filtration system 126 may determine a confidence score associated with each suggestion. The confidence score may be based on a clinical rationale and a strength of a clinical ranking associated with each suggestion. Further, the filtration system 126 may identify, from the suggestions, a subset of the suggestions (e.g., selected suggestions) that satisfy a target confidence score (e.g., a threshold), as suggestions to present to the clinician in connection with the patient, to advance the mental health treatment for the patient. The suggestion filtration system 126, in the illustrative embodiment, stores data indicative of the selected suggestions in the filtration data 136. In some embodiments, the suggestion filtration system 126 may further exclude certain suggestions based on a set of exclusion criteria which may be defined as a set of one or more rules that are specific to a given patient, that, if satisfied by a given suggestion, indicate that the suggestion should be excluded from the set of selected suggestions to be presented to the clinician. The exclusion criteria may be stored in the filtration data 136 in at least some embodiments.

In performing the operations described above, the cloud-based system 110 may utilize one or more artificial intelligence models, such as RNNs, LLMs, or others, that may be stored in a set of artificial intelligence model data 138.

As described in more detail herein, by performing the operations of the audio capture system 120, the transcription system 122, the clinical artifact system 124, and the suggestion filtration system 126, using artificial intelligence, the system 100 vastly increases the efficiency and effectiveness with which clinicians may provide mental health treatment to patients. Accordingly, the system 100 enables clinicians to not only provide treatment to more patients over the same time period, by reducing the burden that would otherwise be imposed by transcribing notes, researching a diagnosis, determining next steps, and re-evaluating patient information in preparation for each upcoming session, the system 100 also provides consistent determinations as to a treatment plan and next steps based on artificial intelligence models that have a been trained on evidence from a large body of outcomes for similarly situated patients.

In the illustrative embodiment, the audio data 130, the transcription data 132, the clinical artifact data 134, the filtration data 136, and the artificial intelligence model data 138 are stored in corresponding cloud-based data stores, such as a combination of relational database and blob storage buckets. However, it should be appreciated that the audio data 130, the transcription data 132, the clinical artifact data 134, the filtration data 136, and the artificial intelligence model data 138 may be stored in any type of data storage capable of storing data received by, used by, and/or generated by the cloud-based system 110. Further, although the audio data 130, the transcription data 132, the clinical artifact data 134, the filtration data 136, and the artificial intelligence model data 138 are represented in FIG. 1 as singular, separate data stores, it should be appreciated that the audio data 130, the transcription data 132, the clinical artifact data 134, the filtration data 136, and the artificial intelligence model data 138 (or portions thereof) may each be stored in multiple data storages in some embodiments.

Although the cloud-based system 110 is described herein in the singular, it should be appreciated that the cloud-based system 110 may be embodied as or include multiple servers/systems in some embodiments. Further, although the cloud-based system 110 is described herein as a cloud-based system, it should be appreciated that the system 110 may be embodied as one or more servers/systems residing outside of a cloud computing environment in other embodiments. In cloud-based embodiments, the cloud-based system 110 may be embodied as a server-ambiguous computing solution similar to that described below.

Each of the user devices 140, 142 may be embodied as any type of device or system capable of interacting with the cloud-based system 110 (e.g., via a network, using one or more corresponding communication protocols, application programming interface (API) calls, etc.) and/or otherwise capable of performing the functions described herein. It should be appreciated that, in some embodiments, each user device 140, 142 may execute an application to interact with the cloud-based system 110, which may be embodied as any type of application suitable for performing the functions described herein. In particular, in some embodiments, the application may be embodied as a mobile application (e.g., a smartphone application), a cloud-based application, a web application, a thin-client application, and/or another type of application. For example, in some embodiments, an application (e.g., executed by a corresponding user device 140, 142) may serve as a client-side interface (e.g., via a web browser) for a web-based application or service (e.g., executed / provided by the cloud-based system 110).

The network 150 may be embodied as any one or more types of communication networks that are capable of facilitating communication between the various devices communicatively connected via the network 150 (e.g., the cloud-system 110 and the user devices 140, 142). As such, the network 150 may include one or more networks, routers, switches, access points, hubs, computers, and/or other intervening network devices. For example, the network 150 may be embodied as or otherwise include one or more cellular networks, telephone networks, local or wide area networks, publicly available global networks (e.g., the Internet), ad hoc networks, short-range communication links, or a combination thereof. In some embodiments, the network 150 may include a circuit-switched voice or data network, a packet-switched voice or data network, and/or any other network able to carry voice and/or data. In particular, in some embodiments, the network 150 may include Internet Protocol (IP)-based and/or asynchronous transfer mode (ATM)-based networks. In some embodiments, the network 150 may handle voice traffic (e.g., via a Voice over IP (VOIP) network), web traffic (e.g., such as hypertext transfer protocol (HTTP) traffic and hypertext markup language (HTML) traffic), and/or other network traffic depending on the particular embodiment and/or devices of the system 100 in communication with one another. In various embodiments, the network 150 may include analog or digital wired and wireless networks. For example, the network 150 may include an IEEE 802.11 network, Public Switched Telephone Network (PSTN), Integrated Services Digital Network (ISDN), Digital Subscriber Line (xDSL) network, mobile telecommunications network, wired Ethernet network, private network (e.g., such as an intranet), radio, television, cable, satellite, and/or any other delivery or tunneling mechanism for carrying data, or any appropriate combination of such networks. The network 150 may enable connections between the various devices/systems 110, 120, 122, 124, 126, 140, 142 of the system 100. It should be appreciated that the various devices/systems 110, 120, 122, 124, 126, 140, 142 may communicate with one another via different networks 150 depending on the source and/or destination devices/systems 110, 120, 122, 124, 126, 140, 142.

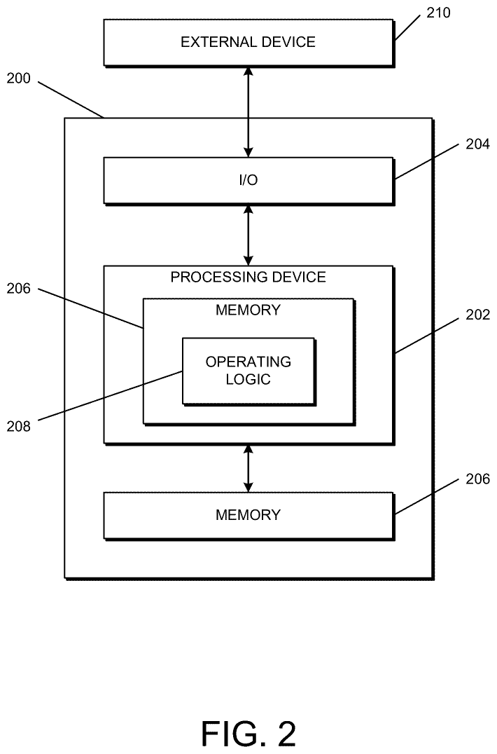

It should be appreciated that each of the cloud-based system 110, the data sets 130, 132, 134, 136, 138 and the user devices 140, 142 may be embodied as, executed by, form a portion of, or associated with any type of device/system, collection of devices/systems, and/or portion(s) thereof suitable for performing the functions described herein (e.g., the computing device 200 of FIG. 2).

Referring now to FIG. 2, a simplified block diagram of at least one embodiment of a computing device 200 is shown. The illustrative computing device 200 depicts at least one embodiment of each of the computing devices, systems, servicers, controllers, switches, gateways, engines, modules, and/or computing components described herein (e.g., which collectively may be referred to interchangeably as computing devices, servers, or systems for brevity of the description). In some embodiments, the computing device 200 may be embodied as a server, desktop computer, laptop computer, tablet computer, notebook, netbook, Ultrabook™, cellular phone, mobile computing device, smartphone, wearable computing device, personal digital assistant, Internet of Things (IoT) device, processing system, wireless access point, router, gateway, and/or any other computing, processing, and/or communication device capable of performing the functions described herein.

The computing device 200 includes a processing device 202 that executes algorithms and/or processes data in accordance with operating logic 208, an input/output device 204 that enables communication between the computing device 200 and one or more external devices 210, and memory 206 which stores, for example, data received from the external device 210 via the input/output device 204.

The input/output device 204 allows the computing device 200 to communicate with the external device 210. For example, the input/output device 204 may include a transceiver, a network adapter, a network card, an interface, one or more communication ports (e.g., a USB port, serial port, parallel port, an analog port, a digital port, VGA, DVI, HDMI, Fire Wire, CAT 5, or any other type of communication port or interface), and/or other communication circuitry. Communication circuitry may be configured to use any one or more communication technologies (e.g., wireless or wired communications) and associated protocols (e.g., Ethernet, Bluetooth®, Wi-Fi®, WiMAX, etc.) to effect such communication depending on the particular computing device 200. The input/output device 204 may include hardware, software, and/or firmware suitable for performing the techniques described herein.

The external device 210 may be any type of device that allows data to be inputted or outputted from the computing device 200. For example, in various embodiments, the external device 210 may be embodied as one or more of the devices/systems described herein, and/or a portion thereof. Further, in some embodiments, the external device 210 may be embodied as another computing device, microphone, printer, display, alarm, peripheral device (e.g., keyboard, mouse, touch screen display, etc.), and/or any other computing, processing, and/or communication device capable of performing the functions described herein. Furthermore, in some embodiments, it should be appreciated that the external device 210 may be integrated into the computing device 200.

The processing device 202 may be embodied as any type of processor(s) capable of performing the functions described herein. In particular, the processing device 202 may be embodied as one or more single or multi-core processors, microcontrollers, or other processor or processing/controlling circuits. For example, in some embodiments, the processing device 202 may include or be embodied as an arithmetic logic unit (ALU), central processing unit (CPU), digital signal processor (DSP), graphics processing unit (GPU), field-programmable gate array (FPGA), application-specific integrated circuit (ASIC), quantum computing processors, and/or another suitable processor(s). The processing device 202 may be a programmable type, a dedicated hardwired state machine, or a combination thereof. Processing devices 202 with multiple processing units may utilize distributed, pipelined, and/or parallel processing in various embodiments. Further, the processing device 202 may be dedicated to performance of just the operations described herein, or may be utilized in one or more additional applications. In the illustrative embodiment, the processing device 202 is of a programmable variety that executes algorithms and/or processes data in accordance with operating logic 208 as defined by programming instructions (such as software or firmware) stored in memory 206. Additionally or alternatively, the operating logic 208 for processing device 202 may be at least partially defined by hardwired logic or other hardware. Further, the processing device 202 may include one or more components of any type suitable to process the signals received from input/output device 204 or from other components or devices and to provide desired output signals. Such components may include digital circuitry, analog circuitry, or a combination thereof.

The memory 206 may be of one or more types of non-transitory computer-readable media, such as a solid-state memory, electromagnetic memory, optical memory, or a combination thereof. Furthermore, the memory 206 may be volatile and/or nonvolatile and, in some embodiments, some or all of the memory 206 may be of a portable variety, such as a disk, tape, memory stick, cartridge, and/or other suitable portable memory. In operation, the memory 206 may store various data and software used during operation of the computing device 200 such as operating systems, applications, programs, libraries, and drivers. It should be appreciated that the memory 206 may store data that is manipulated by the operating logic 208 of processing device 202, such as, for example, data representative of signals received from and/or sent to the input/output device 204 in addition to or in lieu of storing programming instructions defining operating logic 208. As shown in FIG. 2, the memory 206 may be included with the processing device 202 and/or coupled to the processing device 202 depending on the particular embodiment. For example, in some embodiments, the processing device 202, the memory 206, and/or other components of the computing device 200 may form a portion of a system-on-a-chip (SoC) and be incorporated on a single integrated circuit chip.

In some embodiments, various components of the computing device 200 (e.g., the processing device 202 and the memory 206) may be communicatively coupled via an input/output subsystem, which may be embodied as circuitry and/or components to facilitate input/output operations with the processing device 202, the memory 206, and other components of the computing device 200. For example, the input/output subsystem may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations.

The computing device 200 may include other or additional components, such as those commonly found in a typical computing device (e.g., various input/output devices and/or other components), in other embodiments. It should be further appreciated that one or more of the components of the computing device 200 described herein may be distributed across multiple computing devices. In other words, the techniques described herein may be employed by a computing system that includes one or more computing devices. Additionally, although only a single processing device 202, I/O device 204, and memory 206 are illustratively shown in FIG. 2, it should be appreciated that a particular computing device 200 may include multiple processing devices 202, I/O devices 204, and/or memories 206 in other embodiments. Further, in some embodiments, more than one external device 210 may be in communication with the computing device 200.

The computing device 200 may be one of a plurality of devices connected by a network or connected to other systems/resources via a network (e.g., devices of the cloud-based system 110 or, more generally, the system 100). The network may be embodied as any one or more types of communication networks that are capable of facilitating communication between the various devices communicatively connected via the network. As such, the network may include one or more networks, routers, switches, access points, hubs, computers, client devices, endpoints, nodes, and/or other intervening network devices. For example, the network may be embodied as or otherwise include one or more cellular networks, telephone networks, local or wide area networks, publicly available global networks (e.g., the Internet), ad hoc networks, short-range communication links, or a combination thereof. In some embodiments, the network may include a circuit-switched voice or data network, a packet-switched voice or data network, and/or any other network able to carry voice and/or data. In particular, in some embodiments, the network may include Internet Protocol (IP)-based and/or asynchronous transfer mode (ATM)-based networks. In some embodiments, the network may handle voice traffic (e.g., via a Voice over IP (VOIP) network), web traffic, and/or other network traffic depending on the particular embodiment and/or devices of the system in communication with one another. In various embodiments, the network may include analog or digital wired and wireless networks (e.g., IEEE 802.11 networks, Public Switched Telephone Network (PSTN), Integrated Services Digital Network (ISDN), and Digital Subscriber Line (xDSL)), Third Generation (3G) mobile telecommunications networks, Fourth Generation (4G) mobile telecommunications networks, Fifth Generation (5G) mobile telecommunications networks, a wired Ethernet network, a private network (e.g., such as an intranet), radio, television, cable, satellite, and/or any other delivery or tunneling mechanism for carrying data, or any appropriate combination of such networks. It should be appreciated that the various devices/systems may communicate with one another via different networks depending on the source and/or destination devices/systems.

It should be appreciated that the computing device 200 may communicate with other computing devices 200 via any type of gateway or tunneling protocol such as secure socket layer or transport layer security. The network interface may include a built-in network adapter, such as a network interface card, suitable for interfacing the computing device to any type of network capable of performing the operations described herein. Further, the network environment may be a virtual network environment where the various network components are virtualized. For example, the various machines may be virtual machines implemented as a software-based computer running on a physical machine. The virtual machines may share the same operating system, or, in other embodiments, different operating system may be run on each virtual machine instance. For example, a “hypervisor” type of virtualizing is used where multiple virtual machines run on the same host physical machine, each acting as if it has its own dedicated box. Other types of virtualization may be employed in other embodiments, such as, for example, the network (e.g., via software defined networking) or functions (e.g., via network functions virtualization).

Accordingly, one or more of the computing devices 200 described herein may be embodied as, or form a portion of, one or more cloud-based systems (e.g., the cloud-based system 110). In cloud-based embodiments, the cloud-based system may be embodied as a server-ambiguous computing solution, for example, that executes a plurality of instructions on-demand, contains logic to execute instructions only when prompted by a particular activity/trigger, and does not consume computing resources when not in use. That is, system may be embodied as a virtual computing environment residing “on” a computing system (e.g., a distributed network of devices) in which various virtual functions (e.g., Lambda functions, Azure functions, Google cloud functions, and/or other suitable virtual functions) may be executed corresponding with the functions of the system described herein. For example, when an event occurs (e.g., data is transferred to the system for handling), the virtual computing environment may be communicated with (e.g., via a request to an API of the virtual computing environment), whereby the API may route the request to the correct virtual function (e.g., a particular server-ambiguous computing resource) based on a set of rules. As such, when a request for the transmission of data is made by a user (e.g., via an appropriate user interface to the system), the appropriate virtual function(s) may be executed to perform the actions before eliminating the instance of the virtual function(s).

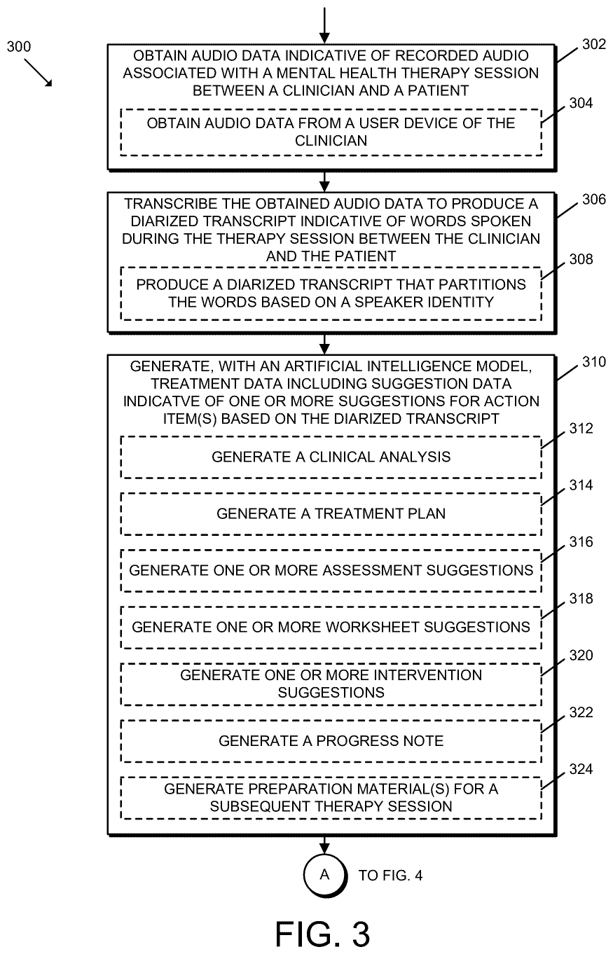

Referring now to FIG. 3, in use, a computing system (e.g., the system 100, including the cloud-based system 110, and/or other computing devices described herein) may execute a method 300 for determining patient-specific therapeutic action items with artificial intelligence. In the illustrative embodiment, it should be appreciated that the method 300 may be executed, in full or in part, by the cloud-based system 110 of the system 100. It should be appreciated that the particular blocks of the method 300 are illustrated by way of example, and such blocks may be combined or divided, added or removed, and/or reordered in whole or in part depending on the particular embodiment, unless stated to the contrary.

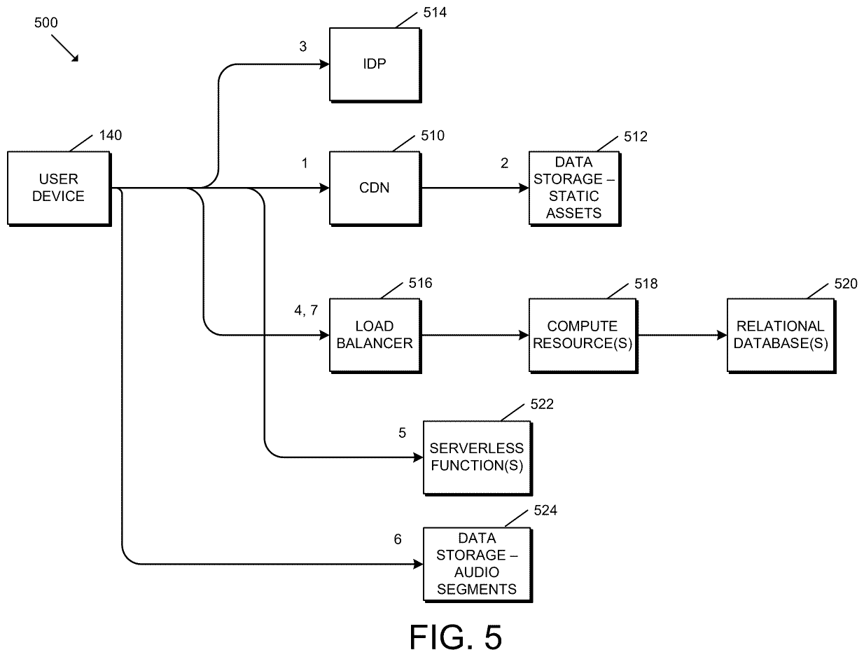

The illustrative method 300 begins with block 302 in which the computing system obtains audio data. The audio data, in the illustrative embodiment, is indicative of recorded audio associated with a mental health therapy session between a clinician and a patient. Further, in doing so, the computing system may obtain the audio data from a user device, such as the user device 140, of the clinician, as indicated in block 304. Referring to FIG. 5, a pipeline 500 that may be used by the computing system for audio capture is shown. The user device 140 associated with a clinician may request an application from a content delivery network (CDN) 510 at a defined network accessible location. In some embodiments, the user device 140 submits the request through a web browser or other application that is capable of rendering text, images, and/or other content for presentation to the user (e.g., the clinician) according to executable instructions (e.g., hypertext markup language (HTML), JavaScript, etc.) provided to the application. The content delivery network 510 may be embodied as a distributed network of servers that store copies of web content (e.g., images, HTML pages, etc.) at various geographic locations, and provides the content to a user through the server(s) closest to the user, to enable enhanced responsiveness to requests and reduced loading times. In at least some embodiments, one or more of the operations of the CDN 510 are performed with Amazon CloudFront. In response to a request, the CDN 510 serves static assets from data storage 512. The static assets are content such as images, text, HTML, JavaScript files and/or other files that do not change as a function of any variables (e.g., are not dynamic). The data storage 512 may be embodied as a blob storage or object storage, in which relatively large amounts of unstructured data is held in non-hierarchical storage areas known as data lakes. In some embodiments, the data storage 512 is a bucket or container for objects in Amazon Simple Storage Service (S3).

Subsequently, in the pipeline for audio capture, the clinician authenticates via the application (e.g., web browser) executed by the user device 140 using an identity provider (IDP) 514. The IDP 514 may be embodied as a system that creates, maintains, and manages identity information for users and provides authentication services within a network. In the illustrative embodiment, the IDP 514 facilitates connections between cloud computing resources and users (e.g., the clinician using the user device 140), to eliminate the need for the user (e.g., clinician) to re-authenticate to every device or resource utilized by the user (e.g., clinician) in the system. In at least some embodiments, one or more of the operations of the IDP 514 are performed with Amazon Cognito or delegates to a third-party SAML-based IDP. Subsequently, the application executed on the user device 140 requests a session to start via an application programming interface (API) call. As part of the operation, the clinician may grant the application (e.g., the web browser) access to the microphone of the user device 140, if the application does not already have access to the microphone. The request via the API call, in the illustrative embodiment, is transmitted to a load balancer 516. In operation, the load balancer 516 distributes traffic, such as requests, evenly across multiple resources to improve fault tolerances to prevent a single resource from becoming overloaded while other resources are underutilized. In the illustrative embodiment, those resources include compute resources 518, such as processor cycles that may be accessed via one or more virtual machine instances. The resources, in the illustrative embodiment, also include a relational database system (RDS) 520. The RDS 520, in the illustrative embodiment, organizes data into one or more tables with rows and columns, in which each piece of data is connected to related data through defined relationships. The architecture of the RDS 520 enables efficient retrieval of information, such as in response to requests from the compute resources 518 in the execution of an application in a virtual machine.

Subsequently, the user device 140 executing the application for the clinician, requests a short-lived tokenized uniform resource locator (URL) to a data storage system 524. In the illustrative embodiment, the data storage 524 is a blob storage bucket in Amazon S3. The request, in the illustrative embodiment, is processed by a serverless function 522 in which resources to execute the function are allocated at the time the function is to be executed, then are immediate deallocated after execution of the function. In at least some embodiments, the serverless function 522 is implemented as an Amazon Lambda function. The clinician application executed by the user device 140 captures audio from a therapy session between the clinician and a patient in segments or chunks of a predefined length. In the illustrative embodiment, the segments are five seconds long. Further, the user device 140 uploads the segments of audio to the data storage 524. In at least some embodiments, the user device 140 creates a local queue (e.g., in the memory 206) to temporarily store the audio segments. From the queue, the user device 140 iteratively and continually uploads the audio segments to the data storage 524, provided that connectivity to the data storage 524 is available. In the event that connectivity is lost, the user device 140 discontinues uploading the audio segments until connectivity is reestablished. By utilizing a local queue as described above, the user device 140 guards against losing audio that is not successfully transmitted from the user device 140 to the data storage 524. At the end of the therapy session, the user device 140 executing the clinician application sends a request via an API call to end the session. The request is sent to the load balancer 516 and ultimately to the compute resources 518, which may be allocated for use in a virtual machine.

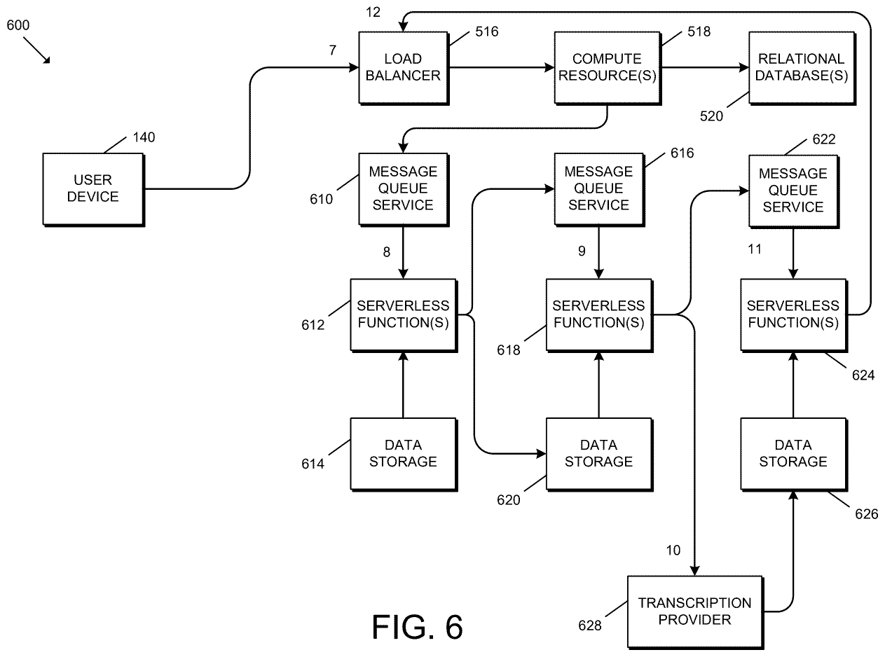

Referring back to FIG. 3, continuing the method 300, the computing system advances to block 306 in which the computing system transcribes the obtained audio data. In doing so, the computing system, in the illustrative embodiment, produces a diarized transcript that is indicative of words spoken during the therapy session between the clinician and the patient. As indicated in block 308, the computing system, in the illustrative embodiment, produces a diarized transcript that partitions (e.g., groups) the word spoken in the therapy session based on the identity of each speaker. That is, the transcript indicates which person spoke which words. Referring now to FIG. 6, the computing system may utilize a pipeline 600 for transcription of the audio recording of the therapy session from block 302 of the method 300.

When the session for recording the audio of the therapy session has ended, the computing system queues a request to combine all audio segments (e.g., the five-second segments) into a single file. The computing system may queue the request using a message queue service 610. The message queue service 610 may be embodied as a distributed message queuing service that enables programmatic sending of messages, such as via web service applications, to enable communication between the applications. In some embodiments, the computing system performs one or more operations of the message queue service using Amazon Simple Queue Service (SQS). In response, a serverless function 612, such as an Amazon Lambda function, reads the audio segments from the data storage 614. The data storage 614 may be embodied as a blob or object data storage, such as an Amazon S3 container or bucket. In some embodiments, the data storage is the data storage 524 described above with reference to the pipeline 500. In the illustrative embodiment, the computing system deletes the audio segments after they have been concatenated into a single file. Doing reduces duplication of data and frees up data storage for other uses.

Subsequently, the computing system utilizes a serverless function 618 to obtain the location (e.g., a URL) of the file in which the audio segments are combined. In the illustrative embodiments, the serverless function 618 obtains the location of the file via a message queue services 616. In at least some embodiments, the message queue service 616 is implemented with Amazon SQS. The file, in the illustrative embodiment, is in the data storage 614. Further, the computing system utilizes the serverless function 618 to call a transcription system 628 and provides a tokenized version of the location (e.g., the URL) of the file (e.g., in the data storage 614) to the transcription system 628. Further, the computing system utilizes the serverless function 618 to provide a tokenized location (e.g., URL) of another data storage, such as the data storage 620, as the target location where the diarized transcript should be stored. The data storage 620, in the illustrative embodiment, is a blob or object data storage, such as an Amazon S3 container or bucket. The transcription system 628 transcribes the audio from the file and returns a diarized transcript. The diarized transcript, in the illustrative embodiment, is encoded as a JavaScript Object Notation (JSON) document and is stored in a data storage 626, which may be a blob or object data storage, such as an Amazon S3 container or bucket.

Additionally, the computing system, upon receipt of the transcript, queues a message to start analyzing the transcript. The computing system utilizes the message queue service 622 (e.g., Amazon SQS) to queue the message and utilizes the serverless function 624 (e.g., an Amazon Lambda function) to analyze the transcript. In the illustrative embodiment, to conserve data storage, the computing system deletes the audio file after the transcript has been analyzed. In analyzing the transcript with the serverless function 624, the computing system may utilize contextual data, as described in more detail below.

Referring back to FIG. 3, continuing the method 300, the computing system generates, with an artificial intelligence model, treatment data. The treatment data, in the illustrative embodiment, includes suggestion data, which may be embodied as any data that is indicative of one or more suggestions. Each suggestion corresponds to an action item to advance the mental health treatment for a corresponding patient. In the illustrative embodiment, the computing system generates the treatment data, including the suggestion data, based on the diarized transcript that was produced in block 306. In doing so, and as indicated in block 312, the computing system may generate a clinical analysis based on the diarized transcript.

Additionally or alternatively, the computing system may generate a treatment plan, as indicated in block 314. The computing system may generate, from the diarized transcript, one or more assessment suggestions, as indicated in block 316. Additionally or alternatively, the computing system may generate one or more worksheet suggestions in block 318. The computing system may also generate one or more intervention suggestions in block 320. In some embodiments, the computing system may generate a progress note in block 322. The computing system may also generate preparation materials to prepare the clinician for a subsequent session with the patient, in block 324.

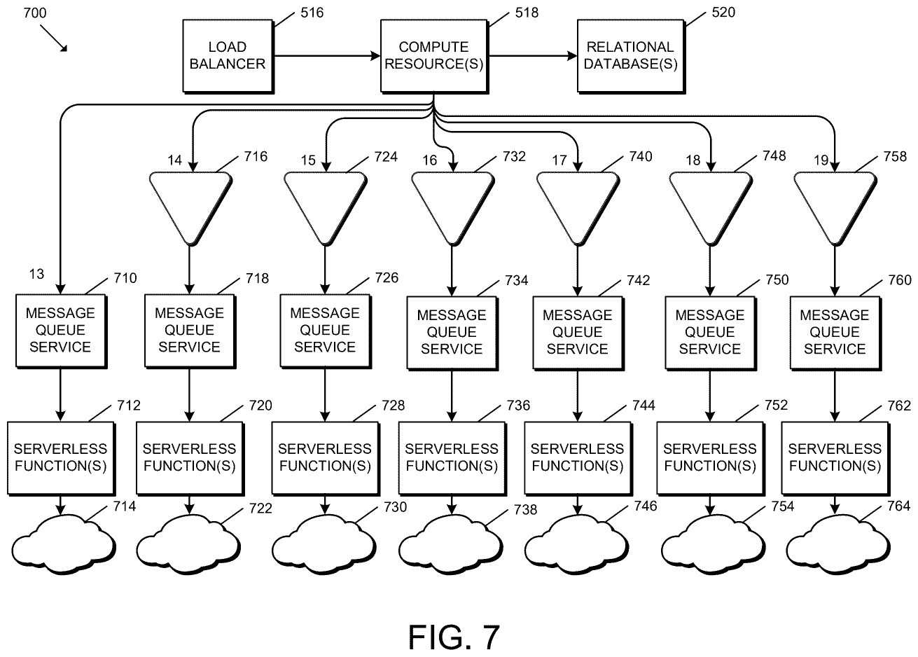

Referring now to FIG. 7, the computing system may utilize a pipeline 700 for progress note and treatment plan generation. That is, the computing system may utilize the pipeline 700 to perform the operations associated with block 310 of the method 300. The computing system may utilize the components 710, 712, 714 of the pipeline 700 to generate a clinical analysis. That is, the computing system may utilize a message queue service 710 to queue a serverless function 712 to generate the clinical analysis. The computing system may utilize the serverless function 712 to generate the clinical analysis from the diarized transcript using a large language model 714 (e.g., from the artificial intelligence model data 138). The large language model 714 may be fine tuned based on data indicative of clinical outcomes across thousands, tens of thousand, hundreds of thousands, or more patients. In the illustrative embodiment, the large language model 714 is fine tuned based on over 3.8 million outcome measures, across over 250,000 clients and over 1,000 organizations. Further, the large language model 714 may utilize over a thousand curated, evidence-based clinical assessments, homework assignments, symptom trackers, and therapeutic interventions.

The computing system may utilize the components 716, 718, 720, 722 to generate a treatment plan. In doing so, the computing system may utilize a message queue service 718 to queue the generation of the treatment plan, in response to a determination 716 that a treatment plan has not already been created for the patient represented in the diarized transcript. The computing system may generate the treatment plan with a serverless function 720. In doing so, the computing system may collect additional patient contextual data, including prior transcripts and/or demographic information associated with the patient. Further, the computing system executes a multi-step workflow with a large language model 722 that creates multiple different types of treatment plans and that can be customized on a per-client basis.

Additionally, the computing system may utilize the components 724, 726, 728, 730 of the pipeline 700, to generate one or more assessment suggestions. In doing so, the computing system may utilize a message queue service 726 to queue a serverless function 728 to generate one or more assessment suggestions. The computing system, in the execution of the serverless function 728, may collect additional patient context information. The additional patient context information may include an accepted treatment plan, the diagnosis and focus of treatment, as approved by the clinician, prior transcripts, and clinical assessments available to the client (e.g., clinician) mapped to the foci of treatment. Further, the computing system, utilizing the components 732, 734, 736, 738 of the pipeline 700, may generate one or more worksheet suggestions. In doing so, the computing system may utilize a message queue service 734 to queue a serverless function 736 to generate worksheet suggestions. That is, the computing system may identify, from the library of worksheets, one or more worksheets that should be completed by the patient. The serverless function 736 may determine the worksheet suggestions based on collecting additional patient context information, which may include an accepted treatment plan and any worksheets that are available to the client (e.g., the clinician).

To generate intervention suggestions, the computing system may utilize the components 740, 742, 744, 746 of the pipeline 700. More specifically, the computing system may utilize a message queue service 742 to queue a serverless function 744 to generate the intervention suggestions. In doing so, the serverless function 744 may collect additional patient context information. The additional patient context information may include an accepted treatment plan, the diagnosis and focus of treatment, as approved by the clinician in the treatment plan, the current and any previous session transcripts, and any data indicative of interventions that have been performed on the patient.

To generate the progress note, the computing system may utilize the components 748, 750, 752, 754 of the pipeline 700. More specifically, the computing system may utilize a message queue service 750 to queue a serverless function 752 to generate the progress note. The serverless function 752 may collect additional patient context information to generate the progress note. The additional patient context information may include any treatment plan associated with the patient, demographic information associated with the patient, clinician preferences, and any special-case context information, which may depend on the type of progress note being generated. In the illustrative embodiment, the process is performed in a multi-step workflow in a large language model orchestration system. The multi-step workflow may create different types of progress notes and may be customized on a per-client (e.g., clinician) basis.

Additionally, the computing system may utilize the components 758, 760, 762, 764 of the pipeline 700 to generate preparation materials for a subsequent therapy session between the clinician and the patient. The computing system may utilize a message queue service 760 to queue a serverless function 762 to generate the preparation materials. In doing so, the serverless function 762 may collect additional patient context information. The additional patient context information includes any treatment plans associated with the patient and demographic data associated with the patient. In the illustrative embodiment, the serverless function 762 utilizes the large language model (e.g., in the artificial intelligence model data 138) that is trained as described above, to produce the preparation materials.

The generation of the clinical analysis, treatment plan, assessment suggestion(s), worksheet suggestion(s), intervention suggestion(s), progress note(s), and preparation material(s) as described above may be performed with an one or more artificial intelligence models 714, 722, 730, 738, 746, 754, 764 (e.g., stored in the artificial intelligence model data 138), such as a large language model. In the illustrative embodiment, the artificial intelligence model(s) 714, 722, 730, 738, 746, 754, 764 may be trained or fine tuned based on data indicative of clinical outcomes across thousands, tens of thousand, hundreds of thousands, or more patients, may be additionally trained or fine tuned based on over 3.8 million outcome measures, across over 250,000 clients and over 1,000 organizations, and may utilize over a thousand curated, evidence-based clinical assessments, homework assignments, symptom trackers, and therapeutic interventions. Though shown as separate components 714, 722, 730, 738, 746, 754, 764, one or more of the artificial intelligence models 714, 722, 730, 738, 746, 754, 764 may be the same artificial intelligence model (e.g., the same large language model) or a combination of artificial intelligence models (e.g., an ensemble of artificial intelligence models) that are trained on different portions of the training data and other data described above.

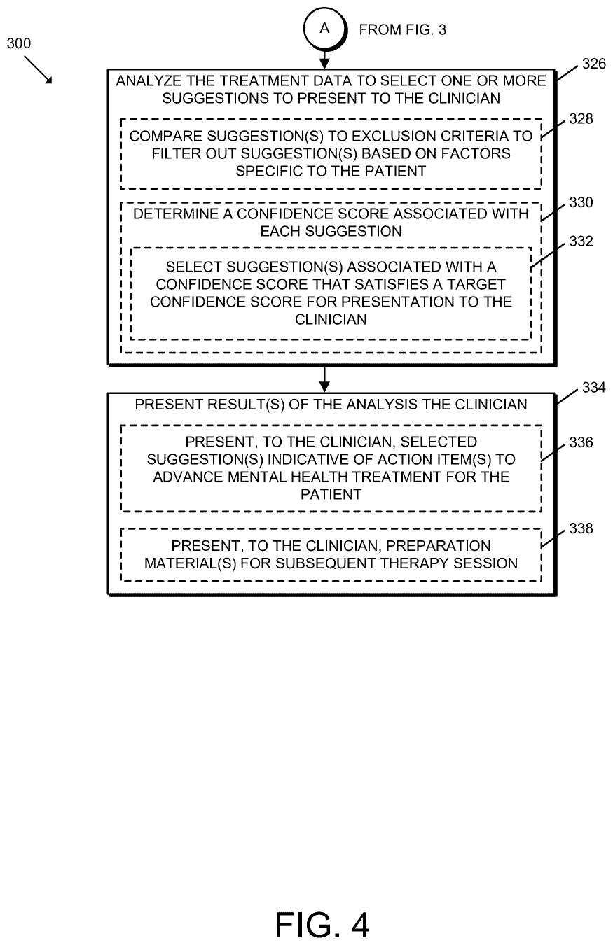

Referring now to FIG. 4, the method 300 continues to block 326 in which the computing system analyzes the treatment data from block 310. In doing so, in block 328, the computing system may compare suggestions, such as assessment suggestions, worksheet suggestions, and/or intervention suggestions to exclusion criteria to filter out (e.g., remove from further analysis) one or more suggestions. The exclusion criteria is based on factors that are specific to the patient associated with the treatment data and the diarized transcript. For example, if the patient has already completed a particular worksheet, the exclusion criteria may indicate to filter out any suggestions to assign that worksheet to the client. Similarly, if a particular assessment suggestion has already been completed relative to the patient, the exclusion criteria may indicated to filter out any suggestions to perform that assessment suggestion. As such, the exclusion criteria, in the illustrative embodiment, is dynamic and changes over time, to filter out different suggestions based on the progress of the patient through a treatment program. The computing system may determine a confidence score associated with each suggestion, as indicated in block 330. That is, the artificial intelligence model, may produce, in addition to a suggestion, a corresponding confidence score associated with that suggestion. The confidence score may be based on internal calculations within the artificial intelligence model, such as an analysis of a probability distribution of possible outputs from the artificial intelligence model. For large language models, the confidence in a particular output may be determined as a log probability, or logarithm of the probability, p, of a token occurring at a particular location based, at least in part, on previous tokens in the context. Given that the probabilities are determined on a logarithmic scale, small differences in log probabilities represent large differences in actual probabilities. Further, when analyzing sequences of tokens, log probabilities can be summed to provide an overall probability or confidence for the sequence of tokens. In block 332, the computing system may select one or more suggestions that have a confidence score that satisfies a target confidence score (e.g., a minimum confidence score, such as 90%). The one or more suggestions having the confidence score that satisfies the target confidence score may be presented to the clinician, such as via the user device 140.

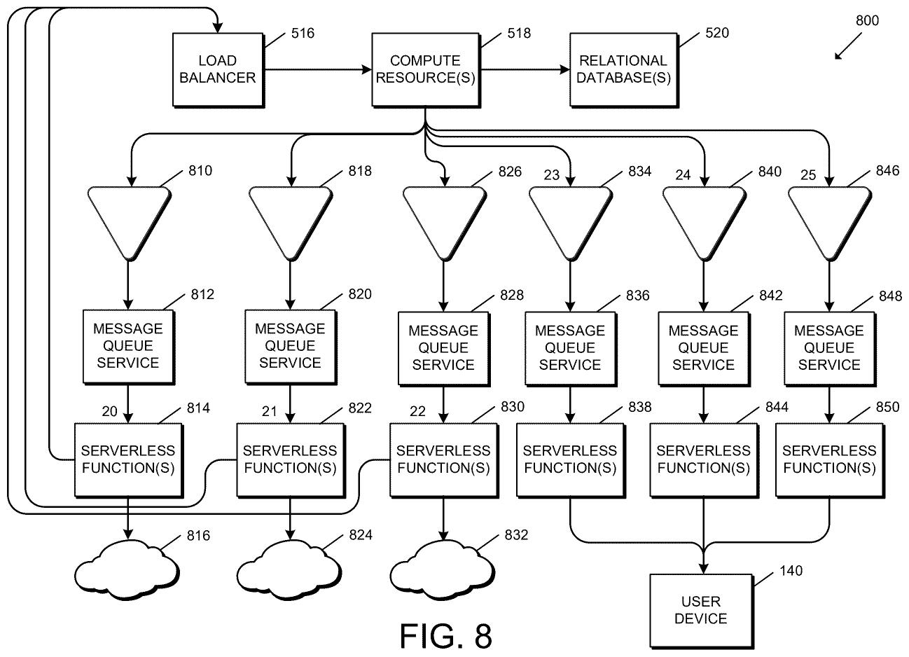

Referring now to FIG. 8, the computing system may utilize a pipeline 800 to select suggestions to present to a clinician. The computing system may utilize a set of components 810, 812, 814, 816 to analyze assessment suggestions. In doing so, the computing system may, after receiving a set of assessment suggestions (e.g., in the treatment data), utilize a message queue service 812 to generate a message to analyze one or more assessment suggestions. In the illustrative embodiment, the computing system analyzes the one or more assessment suggestions with a serverless function 814 and an artificial intelligence model 816, which may be the artificial intelligence model 730 of the pipeline 700. That is, the serverless function 814 may determine the confidence score that the artificial intelligence model 816 assigned to each of the assessment suggestions and determine whether the confidence score satisfies the target confidence score. In the illustrative embodiment, the serverless function 814 performs the analysis further on data indicative of a clinical rationale and a strength of a clinical ranking associated with each assessment suggestion. The clinical rationale and the strength of the clinical ranking may be determined as a function of the training data utilized to train the corresponding artificial intelligence model 816. Similarly, the computing system may utilize a set of components 818, 820, 822, 824 to analyze one or more worksheet suggestions (e.g., from the treatment data described above). In doing so, the computing system may receive a set of one or more worksheet suggestions and, in response, utilize a message queue service 820 to queue a serverless function 822 to analyze the one or more worksheet suggestions. In doing so, the serverless function 822 may determine confidence scores produced by the artificial intelligence model 824, which may be the artificial intelligence model 738 of the pipeline 700, in connection with each of the one or more worksheet suggestions and compare those confidence scores to the target confidence score (e.g., 90%). The serverless function 822 may further perform the determination as to which worksheet suggestions to retain within a set for presentation to the clinician as a function of clinical rationale and a strength of a clinical ranking, which may be based on the training data utilized to train the corresponding artificial intelligence model 824. In the illustrative embodiment, the computing system may exclude, from a set of suggestions to present to the clinician, any worksheet suggestions that do not have a corresponding confidence score that satisfies the target confidence score.

The computing system may utilize a set of components 826, 828, 830, 832 of the pipeline 800 to analyze one or more intervention suggestions from the treatment data produced in block 310 of the method 300. In doing so, the computing system may utilize a message queue service 828 to queue a serverless function 830 to analyze the one or more intervention suggestions. The serverless function 830 may determine, from the artificial intelligence model 832, corresponding confidence scores assigned to each of the one or more intervention suggestions that were produced with the artificial intelligence model 832. The artificial intelligence model 832 may be the artificial intelligence model 746 of the pipeline 700. The serverless function 830 may compare the confidence score associated with each intervention suggestion to the target confidence score (e.g., 90%) to identify a set of one or more intervention suggestions to be presented to the clinician. Similar to the serverless functions 814, 822, the serverless function 830 may perform the determination of which intervention suggestions remain in the set to be presented to the clinician further as a function of data indicative of a clinical rational and strength of a clinical ranking for each intervention suggestion.

The computing system may utilize a set of components 834, 836, 838 to apply exclusion criteria to the assessment suggestions from the remaining set of assessment suggestions from the operations associated with the components 810, 812, 814, 816. In doing so, the computing system may utilize a message queue service 836 to queue a serverless function 838 to apply the exclusion criteria to the remaining set of assessment suggestions. Similarly, the computing system may utilize a set of components 840, 842, 844 to filter any remaining worksheet suggestions from the operations associated with the components 818, 820, 822, 824 by applying the exclusion criteria to those worksheet suggestions. Further, the computing system may utilize a set of components 846, 848, 850 to filter any remaining intervention suggestions in the set determined from the operations associated with the components 826, 828, 830, 832, by applying the exclusion criteria. As described above, the exclusion criteria may change or evolve over time, as the patient progresses through a treatment program. Accordingly, a suggestion that may have not been excluded during one iteration of the operations associated with the pipeline 800 may be excluded during a different iteration of the operations associated with the pipeline 800 at a different time. In addition to excluding any suggestions based on the corresponding exclusion criteria, the serverless functions 838, 844, 850, in the illustrative embodiment, may exclude any suggestions that do not have a predefined clinical strength. For example, in the illustrative embodiment, the serverless functions 838, 844, 850 exclude any suggestions that do not have a clinical strength that is determined to be “high.” Referring back to FIG. 4, the method 300 continues in block 334, in which the computing system presents results of the analysis to the clinician. In doing so, in block 336, the computing system may present, to the clinician, one or more selected suggestions indicative of corresponding action items to advance the mental health treatment for the patient. That is, the computing system presents, via the user device 140, the suggestions that were not filtered out in the operations performed relative to the pipeline 800, discussed above. In block 338, the computing system may present, to the clinician (e.g., via the user device 140), a set of preparation materials for a subsequent therapy session with the patient. The preparation materials may be all or a subset of the preparation materials that were generated in block 324 of the method 300.

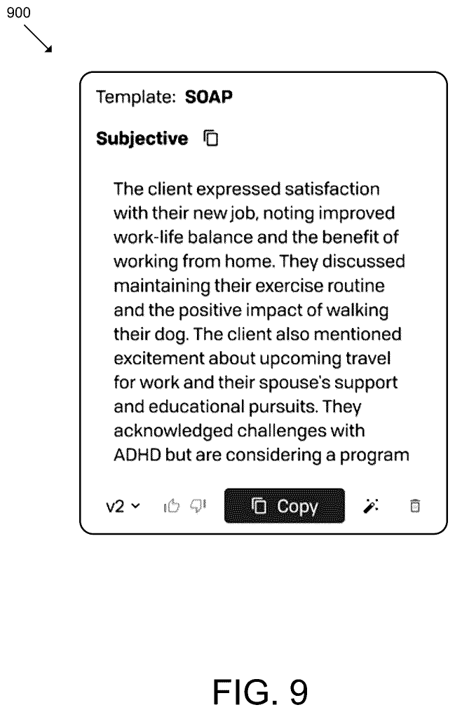

Referring now to FIG. 9, in a user interface 900, the computing system 100 may present a note, such as an intake note or a progress note based on a recording of therapy session between the patient and the clinician. In at least some embodiments, the computing system 100 may generate the note in any of a set of formats, such as SOAP (subjective objective assessment plan), DAP (data assessment and plan), BIRP (behavior intervention response plan) or another format that may be defined by the clinician. Further, the computing system 100 may integrate historical data from treatment plans and past visits (e.g., therapy sessions) to produce notes that are compliant with regulations and are context-aware. In at least some embodiments, the computing system 100 may generate notes in any of multiple languages, such as English, Spanish, or Mandarin.

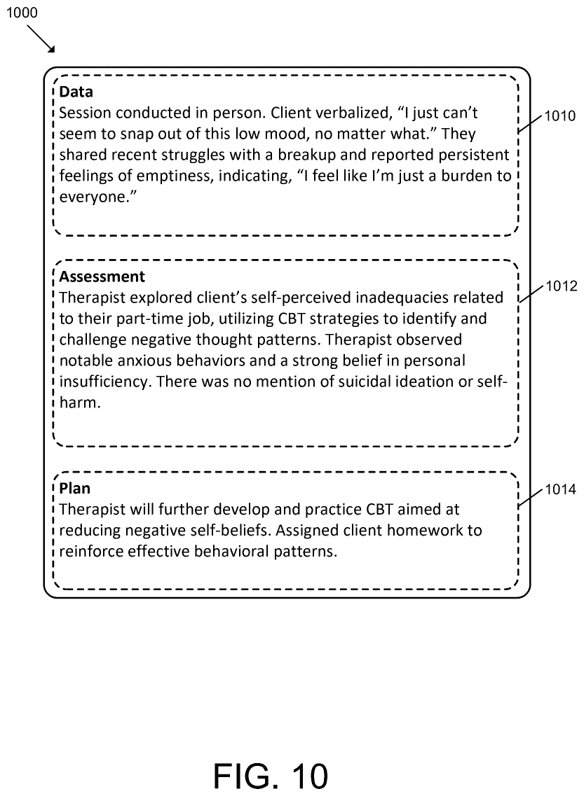

Referring to FIG. 10, in a user interface 1000, the computing system 100 may copy note data directly into an electronic health record associated with the corresponding patient. That is, the computing system 100 may store note data in a digital version of a patient's medical history that may be accessed by multiple healthcare providers having access rights. The note data may include a summary 1010 of a therapy session, based on an audio recording thereof, an assessment 1012 generated by the computing system 100, and a treatment plan 1014 generated by the computing system 100.

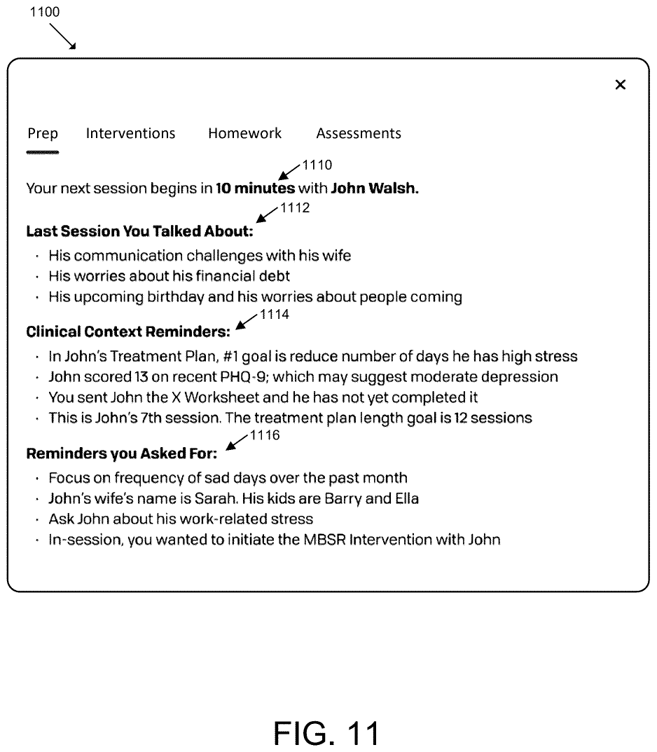

Referring to FIG. 11, in a user interface 1100, the computing system 100 may present preparation materials to the clinician to prepare for a therapy session with a patient. In at least some embodiments, the preparation materials include an amount of time remaining 1110 until the therapy session begins, a summary 1112 of a discussion between the clinician and the patient from the previous therapy session, and a set of clinical context reminders 1114 including a goal of the treatment program, the patient's score on an assessment, any worksheets that the patient has been assigned and the patient's progress relative to those worksheets, the number of sessions that that patient has had with the clinician and the total number of sessions associated with the treatment program. Further, the preparation materials may include reminders 1116 that were identified by the clinician (e.g., via the user device 140), such as action items to address during the upcoming therapy session.

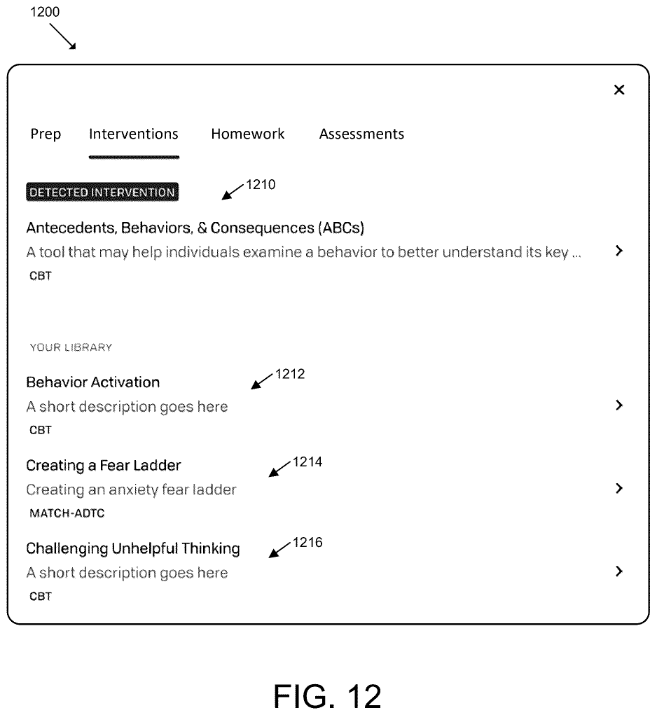

Referring now to FIG. 12, the computing system 100 may present, in a user interface 1200, one or more suggestions for intervention(s). Each intervention represents a structured technique to address both the immediate and long term management of a mental health condition, such as to interfere with and stop or modify a process causing the mental health condition. The set of intervention suggestions in the illustrative embodiment includes an antecedents, behaviors and consequences intervention 1210, a behavior activation intervention 1212, an anxiety feat ladder intervention 1214, and a challenging unhelpful thinking intervention 1216.

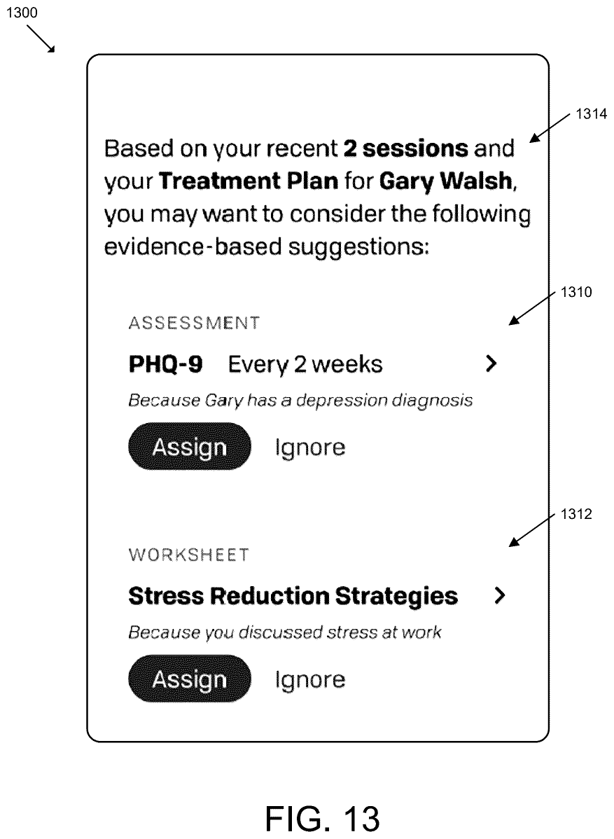

Referring now to FIG. 13, in a user interface 1300, the computing system 100 may present a set of suggestions for use by the clinician in association with a patient. In the illustrative embodiment, the suggestions include an assessment suggestion 1310 and a worksheet suggestion 1312. In at least some embodiments, the computing system 100 may present the suggestions with a description of a context 1314 related to the suggestions to enable the clinician to better understand the rationale associated with the suggestions.

1. A method for determining patient-specific therapeutic action items, the method comprising:
obtaining, by a computing system, audio data indicative of recorded audio associated with a mental health therapy session between a clinician and a patient;
transcribing, by the computing system, the obtained audio data to produce a diarized transcript indicative of words spoken during the therapy session between the clinician and the patient;
generating, by the computing system and with an artificial intelligence model that has been trained using data indicative of patient outcomes, treatment data including suggestion data, indicative of one or more suggestions for corresponding action items to advance mental health treatment for the patient based on the diarized transcript;
analyzing, by the computing system, the treatment data generated by the computing system and with the artificial intelligence model that has been trained using data indicative of patient outcomes to select one or more of the suggestions to present to the clinician to advance the mental health treatment for the patient; and
presenting, by the computing system, the selected one or more suggestions to the clinician to advance the mental health treatment for the patient.

2. The method of claim 1, wherein obtaining audio data comprises obtaining audio data from a user device of the clinician.

3. The method of claim 1, wherein transcribing the obtained audio data comprises producing the diarized transcript that partitions the words spoken during the therapy session based on a speaker identity.

4. The method of claim 1, wherein generating the treatment data comprises generating, with a large language model, a clinical analysis.

5. The method of claim 1, wherein generating the treatment data comprises generating, with a large language model, a treatment plan.

6. The method of claim 1, wherein generating the treatment data comprises generating, with a large language model, one or more assessment suggestions, wherein each assessment suggestion is indicative of an action item relating to an assessment of the patient.

7. The method of claim 1, wherein generating the treatment data comprises generating, with a large language model, one or more worksheet suggestions, wherein each worksheet suggestion is indicative of an action item relating to a worksheet to be completed by the patient.

8. The method of claim 1, wherein generating the treatment data comprises generating, with a large language model, one or more intervention suggestions, wherein each intervention suggestion is indicative of an intervention to be performed relative to the patient.

9. The method of claim 1, wherein generating the treatment data comprises generating a progress note indicative of a progress relative to the mental health of the patient.

10. The method of claim 1, wherein generating the treatment data comprises generating preparation materials for a subsequent therapy session between the clinician and the patient.

11. The method of claim 1, wherein analyzing the treatment data comprises comparing the one or more suggestions to exclusion criteria to filter out at least one of the one or more suggestions based on factors specific to the patient.

12. The method of claim 1, wherein analyzing the treatment data comprises:
determining a confidence score associated with each suggestion; and
selecting at least one of the suggestions associated with a confidence score that satisfies a target confidence score for presentation to the clinician.

13. A computing system for determining patient-specific therapeutic action items, the computing system comprising:
at least one processor; and
at least one memory comprising a plurality of instructions stored thereon that, in response to execution by the at least one processor, causes the computing system to:
obtain audio data indicative of recorded audio associated with a mental health therapy session between a clinician and a patient;
transcribe the obtained audio data to produce a diarized transcript indicative of words spoken during the therapy session between the clinician and the patient;
generate, with an artificial intelligence model that has been trained using data indicative of patient outcomes, treatment data including suggestion data, indicative of one or more suggestions for corresponding action items to advance mental health treatment for the patient based on the diarized transcript;
analyze the treatment data generated with the artificial intelligence model that has been trained using data indicative of patient outcomes to select one or more of the suggestions to present to the clinician to advance the mental health treatment for the patient; and
present the selected one or more suggestions to the clinician to advance the mental health treatment for the patient.

14. The computing system of claim 13, wherein to obtain audio data comprises to obtain audio data from a user device of the clinician.

15. The computing system of claim 13, wherein to transcribe the obtained audio data comprises to produce the diarized transcript that partitions the words spoken during the therapy session based on a speaker identity.

16. The computing system of claim 13, wherein to generate the treatment data comprises to generate, with a large language model, a clinical analysis.

17. The computing system of claim 13, wherein to generate the treatment data comprises to generate, with a large language model, a treatment plan.

18. The computing system of claim 13, wherein to generate the treatment data comprises to generate, with a large language model, one or more assessment suggestions, wherein each assessment suggestion is indicative of an action item relating to an assessment of the patient.

19. The computing system of claim 13, wherein to generate the treatment data comprises to generate, with a large language model, one or more worksheet suggestions, wherein each worksheet suggestion is indicative of an action item relating to a worksheet to be completed by the patient.

20. The computing system of claim 13, wherein to generate the treatment data comprises to generate, with a large language model, one or more intervention suggestions, wherein each intervention suggestion is indicative of an intervention to be performed relative to the patient.
